(19) United States
(12) Patent Application Publication
(10) Pub. No.: US 2006/0193811 A1
(43) Pub. Date: Aug. 31, 2006

(54) Composition serving as a carrier for delivery of active ingredients in cosmetic and dermatological applications

(75) Inventors: William A. Oliver III, Clearwater, FL (US); Adalberto Rivera, Clearwater, FL (US)

Correspondence Address: DENNIS G. LAPOINTE;LAPOINTE LAW GROUP, PL, PO BOX 1294, TARPON SPRINGS, FL, 34688-1294, US

(73) Assignee: Ion Labs, Inc., Clearwater, FL (US)

(21) Appl. No.: 11/356,550

(22) Filed: Feb. 17, 2006

Related U.S. Application Data
(60) Provisional application No. 60/656,591, filed on Feb. 25, 2005.

(51) Int. Cl. A61K 8/81

(52) U.S. Cl. 424/70.16

(57) ABSTRACT

The present invention is for a carrier comprising an oil/water emulsion for delivery of active ingredient in a medium of cream or lotion for skin cosmetic and/or dermatological care.

### RELATED APPLICATION

This application claims the benefit of U.S. provisional application 60/656,591 filed Feb. 25, 2005.

### FIELD OF THE INVENTION

This invention relates to cosmetic and dermatological applications, especially carriers for topical delivery of active ingredients for problems associated with aging skin.

### BACKGROUND OF THE INVENTION

The application works to target the subcutaneous layer for involvement in skin protections, or skin treatments associated with aging skin up to and including reduction of collagen formation, polypeptide elastin degradation, and skin lipid matrix complications.

Human skin is the body's largest organ and it serves us many functions. Covering the entire body, the skins main role is played as a protective and sensory organ. The purpose of the skin is to avert mechanical, thermal, actinic, chemical, and biological stimuli.

The purpose of skin care through cosmetics is to work to strengthen the skin against environmental influences and against loss of endogenous substances, also to assist the horny layer in natural regeneration of current damage.

If barrier properties of the skin are damaged it is vulnerable to absorption of toxic or microbiological invaders are more likely, which could result in damage infection, or even toxic and allergic reactions.

Another aim of skin care is to compensate for loss of sebum or hydration due to constant washing. This section is especially important if natural regeneration is impartial. So basically, skin care products are here to influence protection against environmental factors, as well as to help natural functions move more smoothly or happen at all.

For legal purposes, this oil in water micro-emulsion is intended for use only with cosmetic/dermatological active ingredients in cosmetic products.

Emulsions are usually described as dispersion heterogeneous mixture containing two components that will not usually mix unless established through a common medium. If the two liquids are oil and water, the emulsion will consist of small oil droplets being surrounded by the water droplets when emulsified. So in this case the basic characteristics of the emulsion are determined by the water.

### SUMMARY OF THE INVENTION

The invention is an oil/water micro-emulsion composition serving as a carrier for the effective delivery of active ingredients to localized areas of the skin, wherein topical spreading with the friction caused by agitation helps activate the lipid content of the oil. Furthermore, an important part of the delivery system is the lipid to water ratio. This is even more important than the lipid solubility on the skin.

Because skin does such a great job in protection of the skin to allow electrolytes to pass through, this becomes a factor in the delivery of our active ingredient through the skin. So when we disperse the active ingredient into the water forming solution, a medium is needed in which to protect the ionized solution so its passage through the skin can be allowed. Due to the active ionization of the solution, the oil/water micro-emulsion shields the ionic charge for passage through the membrane.

The aim of our product is to remedy these shortcomings by way the use of the present invention composition that serves as a carrier of medium for the active ingredient.

The composition is an oil/water micro-emulsion carrier for delivering active ingredients for the cosmetic and dermatological care of the skin, the composition further comprises:

an aqueous phase constituent of up to 75% of total weight of the combined carrier constituents, active ingredient constituent and other desired constituents forming the finished cosmetic or dermatological product;

a lipid phase constituent of up to 15% of said total weight;

organic or inorganic humectants or combinations thereof, of up to 15% of said total weight; and

up to 6% of said total weight of constituents comprising any one of an acrylic acid polymer or a multifunctional polymer, polymeric emulsifiers and combinations thereof.

Active ingredients are added to the composition for the intended purpose. Preservatives as well as perfumes may also be added to enhance the shelf life and to make the finish cosmetic product more appealing to the consumer. pH modifiers may also be added as needed. All of these ingredients generally comprise the finished product.

### DETAILED DESCRIPTION OF THE PREFERRED EMBODIMENTS

As mentioned above, the invention is an oil/water micro-emulsion carrier composition for delivering active ingredients for the cosmetic and dermatological care of the skin, the composition comprising:

an aqueous phase constituent of up to 75% of total weight of the combined carrier constituents, active ingredient constituent and other desired constituents forming the finished cosmetic or dermatological product;

a lipid phase constituent of up to 15% of the total weight;

organic or inorganic humectants or combinations thereof, of up to 15% of the total weight; and

up to 6% of the total weight of constituents comprising any one of an acrylic acid polymer or a multifunctional polymer, polymeric emulsifiers and combinations thereof.

The lipid content or constituent of the emulsion is very important and generally comprises about 5-10% total weight of the composition main constituents. Preferably, between 5-8% of lipid content by way of an oil such as avocado oil can be used, due to the high LDL content, with between 36-80% oleic acid, 7-32% palmitic fatty acid, 6-8% linoleic fatty acid, 2-13% palmitoleic fatty acid, and 1.5% steric fatty acid. This being the preferable choice for lipid phase sourcing there are many other options for lipid phase constituents including natural and synthetic oils. For example, the following may be used: mineral oil or waxes, or paraffin oils; any natural oil, including triglycerides such as caprylic or capric acids; fats, waxes and any other natural or synthetic fatty substances; any fatty substance with low carbon number such as 3-36; alkyl benzoates; and silicon oils, phospholipids, or processed hydrocarbons.

The ideal preparation consists of a natural oil, preferably avocado oil, paired with a synthetic organic solvent, preferably propylene glycol or polyethylene glycol (PEG), for dispersion in an aqueous phase before combining with the active ingredient solution.

The aqueous phase of the preparation in this invention should include a diol, or polyol preferably propylene glycol, PEG, or an ethyl glycol, also including one or more polymers up to and including any carbomer, acrylate, acrylamide, copolymer, crosspolymer, carboxy vinyl, or any including a low carbon count Cₙ (where n=3-36) polymer.

Some form of preservative should be added to the aqueous phase in order to ensure microbiological contaminants are kept from forming.

Other lipid phase constituents that can be used are: fats, waxes, oils, and other natural and synthetic fatty substances, preferably fatty acids of alcohols with low carbon numbers, e.g. isopropyl, ethanol, polyethylene glycol, glycerol, or fatty acid esters with alkanoic acids low carbon numbers or with fatty acids, and alcohols, diols, and polyols of low carbon number of 3-36.

Examples below are examples of effective combination of constituents making up a micro-emulsion composition within the scope of the invention, and are not presented to limit the scope of the invention. The numerical values stated represent percentages by weight in formulation based on the total weight of the composition of the carrier micro-emulsion.

### EXAMPLE 1

### Oil/Water Micro-Emulsion

| | |
|---|---|
| Propylene glycol | 10.0 |
| Persea nubigena oil | 10.0 |
| Glycerol | 5.0 |
| Carboxyvinyl polymer | 2.0 |
| Caprylic/Capric Triglyceride | 2.0 |

To the above, water is added to make up the remainder of the composition.

The active ingredient, perfume, preservative, triethanolamine, dyes, anti-oxidant, etc. may be added as desired for the intended application.

### EXAMPLE 2

| | |
|---|---|
| PEG-50 | 8.0 |
| Squalene | 8.0 |
| Acrylate/acrylamide copolymer | 6.0 |
| Mineral oil | 2.0 |
| Glycerol | 2.0 |

To the above, water is added to make up the remainder of the composition. The active ingredient, perfume, preservative, triethanolamine, dyes, anti-oxidant, etc. may be added as desired for the intended application.

### EXAMPLE 3

| | |
|---|---|
| Ethanol | 10.0 |
| Primrose oil | 8.0 |
| C (10-36) alkyl benzoate | 3.0 |
| Glycerol | 5.0 |
| Dimethicone copolyol | 2.0 |

To the above, water is added to make up the remainder of the composition. The active ingredient, perfume, preservative, triethanolamine, dyes, anti-oxidant, etc. may be added as desired for the intended application.

What is claimed is:

1. An oil/water micro-emulsion carrier composition for delivering active ingredients for the cosmetic and dermatological care of the skin, the composition comprising:
an aqueous phase constituent of up to 75% of total weight of the combined carrier constituents, active ingredient constituent and other desired constituents forming the finished cosmetic or dermatological product;
a lipid phase constituent of up to 15% of said total weight;
an organic humectant constituent, inorganic humectant constituent or a combination thereof, of up to 15% of said total weight; and
up to 6% of said total weight of constituents comprising any one of an acrylic acid polymer or a multifunctional polymer, polymeric emulsifiers and combinations thereof.

2. The composition according to claim 1, wherein the lipid phase constituent comprises about 5-15% of the total weight; the humectants comprise about 6-15% of the total weight; the any one of the acrylic acid polymer or the multifunctional polymer, polymeric emulsifiers and combinations thereof comprise about 2-6% of the total weight.

3. The composition according to claim 1, wherein the lipid phase constituent comprises about 6-9% of the total weight; the humectants comprise about 6-9% of the total weight; and the any one of the acrylic acid polymer or the multifunctional polymer, polymeric emulsifiers and combinations thereof comprise about 3-5% of the total weight.

4. The composition according to claim 1, further comprising the addition of a suitable amount of preservative constituent to ensure microbiological contaminants are kept from forming.

5. The composition accounting to claim 1, further comprising the addition of a desired amount of active ingredient constituent designed for cosmetic and/or dermatological care of the skin.

6. The composition accounting to claim 1, further comprising the addition of a desired amount of anti-oxidant consituents.

7. The composition accounting to claim 1, further comprising the addition of a desired amount of a triethanolamine constituent.

8. The composition accounting to claim 1, further comprising the addition of a desired amount of a dye constituent.

9. The composition accounting to claim 1, wherein the lipid constituent is selected from the group of natural and/or synthetic oils comprising mineral oil or waxes, paraffin oils, any natural oil, including triglycerides such as caprylic or capric acids, fats, waxes and any other natural or synthetic fatty substances, any fatty substance with low carbon number such as 10-36; alkyl benzoates, and silicon oils, phospholipids, or processed hydrocarbons, and any combination thereof.

10. The composition accounting to claim 1, wherein the lipid constituent is a natural oil paired with a synthetic organic solvent for dispersion in the aqueous phase constituent before combining with an active ingredient solution.

11. The composition accounting to claim 10, wherein the lipid constituent is avocado oil.

12. The composition accounting to claim 10, wherein synthetic organic solvent is propylene glycol or polyethylene glycol (PEG).

13. The composition according to claim 1, wherein aqueous phase constituent includes a diol, a polyol, propylene glycol, PEG, an ethyl glycol or a combination thereof.

14. The composition according to claim 13, further comprising one or more polymers including any carbomer, acrylate, acrylamide, copolymer, crosspolymer, carboxy vinyl, any polymer having a low carbon count Cₙ (where n=10-36), or any combination thereof.

15. The composition according to claim 1, wherein the lipid constituent comprises one of more of the following: fats, waxes, oils, natural and synthetic fatty substances including fatty acids of alcohols with low carbon numbers between 3-36, including isopropyl, ethanol, polyethylene glycol, glycerol, or fatty acid esters with alkanoic acids low carbon numbers between 3-36 or with fatty acids, and alcohols, diols, and polyols with low carbon numbers between 3-36.

16. The composition according to claim 1, wherein the aqueous phase constituent is water.

17. The composition according to claim 1, wherein the aqueous phase constituent includes one of a diol, polyol preferably propylene glycol, PEG, an ethyl glycol, one or more polymers including any carbomer, acrylate, acrylamide, copolymer, crosspolymer, carboxy vinyl, or any polymer having a low carbon count Cₙ (where n=3-36), or any combination thereof.
